(19) United States
(12) Patent Application Publication
(10) Pub. No.: US 2006/0264698 A1
(43) Pub. Date: Nov. 23, 2006

(54) Mesh implant

(75) Inventors: Thalia Kondonis, Gerrards Cross (GB); Tim Coutts, Cheshire (GB)

Correspondence Address: UNITED STATES SURGICAL,;A DIVISION OF TYCO HEALTHCARE GROUP LP, 195 MCDERMOTT ROAD, NORTH HAVEN, CT, 06473, US

(73) Assignee: Sherwood Services AG

(21) Appl. No.: 11/386,597

(22) Filed: Mar. 22, 2006

Related U.S. Application Data
(60) Provisional application No. 60/664,312, filed on Mar. 22, 2005.

(51) Int. Cl. A61F 2/02

(52) U.S. Cl. 600/37; 128/898; 623/23.74

(57) ABSTRACT

A mesh implant is disclosed which may be utilized for treating urinary incontinence, hernias, uterovaginal prolapses and other related injuries.

### CROSS-REFERENCE TO RELATED APPLICATIONS

This application claims the benefit of U.S. Provisional Patent Application No. 60/664,312 filed Mar. 22, 2005, the entire disclosure of which is hereby incorporated by reference herein.

### BACKGROUND

1. Technical Field

The present disclosure relates to medical implants. More particularly, the present disclosure relates to medical implants having a mesh configuration that are useful for treating urinary incontinence and other related injuries, including vaginal prolapse.

2. Background of Related Art

In women, incontinence, or the inability to control the outflow of urine, can have a variety of causes in the urinary system including congenital defects and defects from trauma or disease. The most common cause of female incontinence is known as stress incontinence and results from weakness or relaxation of supportive tissues and ligaments surrounding the urethra.

Many procedures, several involving urethrovesical elevation, have been devised over the years to remedy urinary stress incontinence. One early procedure involved fixation of the urethrovesical junction to the symphysis pubis by placing sutures through part of the urethral wall, but caused urethral distortion. A modified version of the procedure involved suturing the urethral lumen directly to the symphysis pubis, and placing additional sutures through the bladder. This technique however, often led to urine loss and/or the formation of bladder stones.

An alternative approach involved attaching the urethrovesical junction to the narrow band of strong aponeurotic fibers which extends laterally along the pectineal line of the pubis commonly referred to as Cooper's ligament. In this procedure, which is described in U.S. Pat. No. 5,149,329 to Richardson, the urethrovesical junction is elevated by bringing the paravaginal fascia into juxtaposition with Cooper's ligament through suture placement.

A number of other procedures for urethrovesical elevation involve anchoring the paravaginal fascia to the abdominal wall. See, for example, U.S. Pat. No. 5,112,344 to Petros, which describes looping a filamentary element between the vaginal wall and the rectus abdominis in the anterior wall of the abdomen passing to each side of the urethra to correct the spacial relationship to the pubis.

A sling procedure is disclosed in U.S. Pat. No. 5,013,292 to Lemay and describes a method for correcting female urinary incontinence by implanting a sling-like anchoring device in the skin above the symphysis pubis to adjust the urethrovesical angle. The anchoring device includes a pair of implants each having a head portion adapted to rest on the symphysis pubis and a suture portion connected to the head portion. The head portion is shaped as a figure eight having a central crossbar about which a central portion of the suture is wrapped. Utilizing a bendable needle inserted through the vaginal mucosa, the head portion of each implant is embedded in the skin over the symphysis pubis and the sutures are tied together to support the urethrovesical junction. Alternatively, the ends of the sutures can be tied to a saddle member configured to support the bladder neck.

Slings used for pubovaginal procedures differ in the type of implantable material utilized to produce the sling and anchoring methods. In some cases, the sling is placed under the bladder neck and secured via suspension sutures to a point of attachment (e.g. bone) through an abdominal and/or vaginal incision.

The TVT Tension-free Vaginal Tape procedure utilizes a knitted PROLENE® nonabsorbable, polypropylene mesh. The mesh is a substantially flat, rectangular woven article. The mesh includes a plurality of holes that are ideally sized to promote tissue ingrowth, and to promote bacterial clearance where necessary. A removable plastic sheath surrounds the mesh and is used during insertion of the mesh. Two curved, needle-like elements are each connected to an end of the vaginal sling mesh. A sling-free end of one of the needle-like elements is initially pushed through the vaginal incision and into the paraurethral space. Using a handle attached to the needle, the needle is angulated laterally (for example, to the right) to perforate the endopelvic fascia, then it is angulated in a caudad direction parallel to the midline, guided through the retropubic space and passed through the abdominal incision. The handle is disconnected and the needle is then withdrawn through the abdominal wall, thereby threading a portion of the sling through the tissue of the patient. The handle is then connected to the other needle and the technique is repeated on the contralateral side, so that the mesh sling is looped beneath the bladder neck or urethra, thereby providing appropriate support to the bladder neck or urethra. Once the sheath is removed from the mesh of the TVT product, friction between the mesh and tissue keeps the mesh in position and it becomes very difficult to subsequently adjust the position of the mesh relative to tissue.

The suitable location of an implant is to support the urethra during periods of increased abdominal pressure, but such that the implant does not pull on the urethra during periods of normal abdominal pressure and cause discomfort. This is difficult for surgeons to achieve. Conventional monofilament tape implants are generally very stretchy and surgeons are required to position the tape in the body such that in use, during periods of normal abdominal pressure, the implant is in a stretched or extended position. As the tapes utilized to date have been made of polypropylene (a material that exhibits memory), the tape is stretched when positioned at surgery but subsequently returns to what approaches its original length, i.e., it shortens due to the memory effect.

With respect to sling procedures, if the sling mesh is too loosely associated with its intended physiological environment, the mesh may be ineffective in supporting the urethra and treating incontinence. However, if a mesh is too tightly placed complications such as post-operative urinary retention, sling erosion into the urethra and other damage to surrounding tissue such as the urethra and vagina can occur.

Surgical approaches to applying tension or slack in a sling procedure vary widely. Improper sling tension or sling suture tension can result in increased lateral movement and momentum of the support structures or mesh sling when they are moved due to intra-abdominal pressures. Because many slings are anchored at anatomical positions remote from the urethra, proper tension in a sling is a difficult objective to achieve. Results can vary widely.

Thus, improvements to surgical implants such as sling meshes used to treat urinary incontinence remain desirable.

### SUMMARY

The present disclosure pertains to a novel medical implant made of strands wherein the medical implant has a maximum residual mass density of about 30 g/m² to about 60 g/m², and pores of from about 200 microns to about 2000 microns in diameter in the mesh. In embodiments, the medical implant may have a thickness from about 0.2 mm to about 0.4 mm and a bioactive coating thereon.

The disclosure further describes the use of the above surgical mesh implant in combination with one or more surgical fasteners and the use of such implant to remedy various medical conditions, including urinary incontinence and vaginal prolapse.

In some embodiments, methods for treating urinary incontinence and/or vaginal prolapse include transvaginally introducing a mesh implant having a maximum residual mass density of about 30 g/m² to about 60 g/m², pores of from about 200 microns to about 2000 microns in diameter in the mesh, and a thickness of about 0.2 mm to about 0.4 mm into a patient's body; advancing at least two fixation devices through the vaginal mucosa and into an internal support structure or tissue; and attaching the fixation devices to the patient's internal support tissue, thereby retaining the mesh implant in a position capable of supporting the bladder neck or urethra.

In other embodiments, urinary incontinence and/or vaginal prolapse may be remedied by utilizing a surgical device to introduce the mesh implant of the present disclosure. This procedure includes providing a mesh implant comprising strands having a maximum residual mass density of about 30 g/m² to about 60 g/m², pores of from about 200 microns to about 2000 microns in diameter in the mesh, and a thickness of about 0.2 mm to about 0.4 mm and providing a surgical device having an outer tubular member including a longitudinal proximal end and a curved distal end and a stylet movable within the tubular member and configured to hold an end of the length of material. The stylet is positioned within the tubular member. A transvaginal incision and another skin incision located lateral to the vulva and above the obturator foramen is made, and the curved distal end of the surgical device is passed through the incision over the obturator foramen. The surgical device is manipulated so that the curved distal end passes through the obturator foramen and out the vaginal incision. A proximal end of the stylet is engaged with a first end of the mesh implant, and the stylet is drawn through the tubular member to draw a portion of the mesh implant from the incision, or in the reverse direction made possible, in embodiments, by reversing the stylet. Depending on the configuration of the mesh implant, there may be more than one incision over the obturator foramen on either side, and more than one arm of the mesh may be drawn from the vaginal incision and pulled through the incisions above the obturator foramen on either side.

### BRIEF DESCRIPTION OF THE DRAWINGS

FIGS. 1A, 1B and 1C illustrate surgical meshes according to the present disclosure having various pore configurations;

FIGS. 2A and 2B illustrate surgical meshes according to the present disclosure having fixation devices attached to the ends thereof; and

FIG. 3 illustrates a surgical mesh according to the present disclosure having a trapezoidal configuration and attachment arms.

### DETAILED DESCRIPTION

According to the present disclosure there is provided a surgical implant suitable for use as a sling in a procedure to treat urinary incontinence. The implant includes a mesh, typically in a sling or tape configuration, made of a biocompatible material. The mesh implant typically has a maximum residual mass density of about 30 g/m² to about 60 g/m². The residual mass density is the mass density of the mesh after implantation and the absorption of any bioabsorbable coatings.

The mesh implant of the present disclosure is made of strands which, in turn, may be made of filaments of any suitable biocompatible material. Suitable materials from which the mesh can be made should have the following characteristics: biocompatibility; sufficient tensile strength to support the urethra or bladder neck for treating urinary incontinence; sufficiently inert to avoid foreign body reactions when retained in the human body for long periods of time; exhibit minimal allergic and/or inflammatory response; non-carcinogenic; easily sterilized to prevent the introduction of infection when the mesh is implanted in the human body; minimal elasticity; minimal shrinkage; and have suitably easy handling characteristics for placement in the desired location in the body.

In some embodiments the filaments may be made of a plastic or similar synthetic non-absorbable material. Some examples include polyolefins, such as polyethylene, polypropylene, copolymers of polyethylene and polypropylene, and blends of polyethylene and polypropylene. Polypropylene can be utilized in a particularly useful embodiment.

In another embodiment the filaments of the mesh may be made of an absorbable material such as a polyester. Some specific examples of suitable absorbable materials which may be utilized to form the filaments include trimethylene carbonate, caprolactone, dioxanone, glycolic acid, lactic acid, glycolide, lactide, homopolymers thereof, copolymers thereof, and combinations thereof.

It can be appreciated that filaments which are made in part of an absorbable material may, if desired, enable the implant to have minimal mass following implantation in the body.

In yet another embodiment, the mesh implant may be made of a material that has memory. A mesh with memory urges the surgical implant to adopt a flat conformation.

Typically the mesh comprises strands and includes pores and minute openings, the pores existing between the strands and the minute openings formed within the strands. While the strands of the mesh implant may be formed from more than one filament, in particularly useful embodiments the mesh implant of the present disclosure is formed from one filament, i.e., a monofilament, which is arranged to form loops that give rise to minute openings in the strands; the strands are then woven to produce the mesh utilized in the implant of the present disclosure.

The filaments utilized to produce the strands of the mesh implant may have a diameter of from about 0.02 mm to about 0.15 mm, in embodiments from about 0.08 mm to about 0.1 mm.

The strands may be spaced apart to form pores of from about 200 microns to about 2000 microns in diameter in the mesh, in embodiments from about 500 microns to about 1500 microns in diameter, in other embodiments from about 750 microns to about 1250 microns in diameter. The weave and the density of the strands forming the mesh provide the mesh implant with its necessary strength. A mesh in accordance with the present disclosure has the advantage of maintaining sufficient tensile strength to securely support the urethra or bladder neck where the mesh implant is utilized to treat urinary incontinence (or any other defect or tissue being repaired by the mesh implant). Moreover, due to the specific pore size and the type of weave of the mesh of the present disclosure, the mesh has minimal elasticity and shrinkage, rendering it capable of withstanding periods of increased abdominal pressure which accompany activities such as coughing or sneezing and maintaining its original shape after being subjected to such a stress or strain, especially during the period following initial implantation before any fibrous ingrowth has occurred.

The mesh of the present disclosure also possesses means for promoting tissue ingrowth to create stronger supporting tissue (scar formation). In some embodiments, it may be desirable to provide minute openings in the strands of the mesh to aid tissue ingrowth and to which tissue may more easily adhere.

In general, at least one filament is interwoven or knitted to produce strands of the mesh comprising minute openings. In some embodiments, two filaments may be used to form minute openings in the strands of the mesh which aid tissue ingrowth. However, if one filament can be suitably knotted or twisted to form minute openings of suitable dimensions, this single filament may be used to similar effect to form the strands of the mesh. The minute openings of the strands are typically of a size that permit fibroblast through-growth and ordered collagen laydown, resulting in integration of the mesh into the body. For example, the woven/knitted filaments create minute openings in the strands that may be from about 200 μm to about 1,000 μm in diameter, in embodiments from about 700 μm to about 900 μm in diameter. Rings or loops of material comprising minute openings that are greater than about 200 μm in diameter may be adhered to or formed on the strands of the mesh to provide additional minute openings.

Once produced, the strands may be warp knit or woven into a variety of different mesh shapes. In some embodiments the strands may be arranged to form a net mesh which has isotropic or near isotropic tensile strength and elasticity.

Due to the variability in patient morphology and anatomy, the implant may be of any suitable size. The surgical mesh implant may have a width from about 1 mm to about 50 mm and a length from about 1 mm to about 1000 mm, in embodiments a width from about 3 mm to about 20 mm and a length from about 100 mm to about 750 mm, typically a width from about 6 mm to about 10 mm and a length from about 400 mm to about 600 mm. In one embodiment, a mesh implant of the present disclosure may be in a tape configuration having a width of about 8 mm and a length of about 500 mm.

The shape of the mesh implant of the present disclosure may be varied depending upon the condition to be treated with the mesh implant. Thus, in addition to a tape configuration as described above, the mesh implant may also be circular, rectangular, trapezoidal, etc. For example, in one embodiment the mesh implant of the present disclosure could have a rectangular or trapezoidal shape and may be utilized to treat a cystocele.

The thickness of the surgical mesh of the present disclosure may also vary, but is typically less than about 0.5 mm. In some embodiments, the thickness of the mesh can be from about 0.2 mm to about 0.4 mm.

As noted above, the mesh implants of the present disclosure have a maximum residual mass density of from about 30 g/m² to about 60 g/m², in embodiments from about 45 g/m² to about 60 g/m².

In one embodiment of the present disclosure, filaments may be formed from polypropylene having a diameter of from about 0.07 mm to about 0.1 mm, wherein the strands making up the mesh are spaced to form pores in the mesh of from about 200 μm to about 1000 μm.

In other embodiments, filaments may be formed from polyester having a diameter of from about 0.05 mm to about 0.09 mm, wherein the strands are spaced to form pores in the mesh of about 200 μm to about 500 μm.

In embodiments, the mesh implant of the present disclosure may possess a bioactive coating having at least one bioactive agent. The term “bioactive agent”, as used herein, is used in its broadest sense and includes any substance or mixture of substances that have clinical use. Consequently, bioactive agents may or may not have pharmacological activity per se, e.g., a dye. Alternatively, a bioactive agent could be any agent which provides a therapeutic or prophylactic effect; a compound that affects or participates in tissue growth, cell growth, and/or cell differentiation; a compound that may be able to invoke a biological action such as an immune response; or a compound that could play any other role in one or more biological processes.

Examples of classes of bioactive agents which may be utilized in accordance with the present disclosure include antimicrobials, analgesics, antiadhesive agents, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used.

Suitable antimicrobial agents which may be included as a bioactive agent in the bioactive coating of the present disclosure include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B may be included as a bioactive agent in the bioactive coating of the present disclosure.

Other bioactive agents which may be included as a bioactive agent in the composition of the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the bioactive coating of the present disclosure include viruses and cells, peptides, polypeptides and proteins, analogs, muteins, and active fragments thereof, such as immunoglobulins, antibodies, beta glycans, cytokines (e.g. lymphokines, monokines, chemokines), blood clotting factors, hemopoietic factors, interleukins (IL-2, IL-3, IL-4, IL-6), interferons (β-IFN, (α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; growth factors (e.g., nerve growth factor, insulin-like growth factor); protein inhibitors, protein antagonists, and protein agonists; nucleic acids, such as antisense molecules, DNA and RNA; oligonucleotides; and ribozymes.

A single bioactive agent may be utilized to form the bioactive coating of the mesh implant of the present disclosure or, in alternate embodiments, any combination of bioactive agents may be utilized to form the bioactive coating of the mesh implant of the present disclosure.

A bioactive coating may be applied to the mesh as a composition containing one or more bioactive agents, or bioactive agent(s) dispersed in a suitable biocompatible solvent. Suitable solvents for particular bioactive agents are within the purview of those skilled in the art. In other embodiments, the bioactive coating may include a bioactive agent in a bioabsorbable material.

Absorbable materials which may be utilized to form the bioactive coating of the present disclosure include soluble hydrogels such as gelatin or a starch, or cellulose-based hydrogels. In other embodiments, the absorbable material may be an alginate or hyaluronic acid. Other examples of absorbable materials which may be utilized to form the bioactive coating include trimethylene carbonate, caprolactone, dioxanone, glycolic acid, lactic acid, glycolide, lactide, homopolymers thereof, copolymers thereof, and combinations thereof. The bioactive coating may have any thickness or bulk and may be utilized to provide the mesh implant with suitable handling characteristics. In embodiments, the coating may be in the form of a sheet having a thickness greater than that of the mesh.

In some embodiments, the bioactive coatings of the present disclosure may also include a fatty acid component that contains a fatty acid, a fatty acid salt, or a salt of a fatty acid ester. Suitable fatty acids may be saturated or unsaturated, and include higher fatty acids having more than about 12 carbon atoms. Suitable saturated fatty acids include, for example, stearic acid, palmitic acid, myristic acid and lauric acid. Suitable unsaturated fatty acids include oleic acid, linoleic acid, and linolenic acid. In addition, an ester of fatty acids, such as sorbitan tristearate or hydrogenated castor oil, may be used.

Suitable fatty acid salts include the polyvalent metal ion salts of C₆ and higher fatty acids, particularly those having from about 12 to 22 carbon atoms, and mixtures thereof. Fatty acid salts including the calcium, magnesium, barium, aluminum, and zinc salts of stearic, palmitic and oleic acids may be useful in some embodiments of the present disclosure. Particularly useful salts include commercial “food grade” calcium stearate which includes a mixture of about one-third C₁₆ and two-thirds C ₁₈ fatty acids, with small amounts of the C₁₄ and C₂₂ fatty acids.

Suitable salts of fatty acid esters which may be included in the compositions of the present disclosure include calcium, magnesium, aluminum, barium, or zinc stearoyl lactylate; calcium, magnesium, aluminum, barium, or zinc palmityl lactylate; calcium, magnesium, aluminum, barium, or zinc olelyl lactylate; with calcium stearoyl-2-lactylate (such as the calcium stearoyl-2-lactylate commercially available under the tradename VERV from American Ingredients Co., Kansas City, Mo.) being useful in some embodiments. Other fatty acid ester salts which may be utilized include lithium stearoyl lactylate, potassium stearoyl lactylate, rubidium stearoyl lactylate, cesium stearoyl lactylate, francium stearoyl lactylate, sodium palmityl lactylate, lithium palmityl lactylate, potassium palmityl lactylate, rubidium palmityl lactylate, cesium palmityl lactylate, francium palmityl lactylate, sodium olelyl lactylate, lithium olelyl lactylate, potassium olelyl lactylate, rubidium olelyl lactylate, cesium olelyl lactylate, and francium olelyl lactylate.

Where utilized, the amount of fatty acid component can be in an amount from about 5 percent to about 50 percent by weight of the total bioactive coating, in embodiments from about 10 percent to about 20 percent by weight of the total bioactive coating.

Any coating composition containing the bioactive agent may encapsulate an entire filament, strand or mesh. Alternatively, the bioactive coating may be applied to one or more sides of a filament, strand or mesh. Such a coating will improve the desired therapeutic characteristics of the mesh.

The bioactive coating may be applied to the mesh implant utilizing any suitable method known to those skilled in the art. Some examples include, but are not limited to, spraying, dipping, layering, calendaring, etc. The bioactive agent or bioactive coating may also be incorporated into the absorbable coatings described herein and applied to the mesh implant accordingly.

In some embodiments, the bioactive coating may add bulk to the mesh such that it is easier to handle. Where the bioactive coating includes a bioabsorbable material, the coating should be released into the body after implantation and therefore should not contribute to the foreign body mass retained in the body. Thus, the advantages of a surgical implant having minimal mass are retained.

Where the bioactive coating includes an absorbable material, the coating may be released into the body within a period of time from about 2 days to about 14 days following implantation. In one embodiment the coating may be released within about 2 days to about 3 days following implantation. In another embodiment, the coating may be released within about 7 days to about 14 days following implantation.

The rate of release of a bioactive agent from the bioactive coating on a mesh of the present disclosure can be controlled by any means within the purview of one skilled in the art. Some examples include, but are not limited to, the depth of the bioactive agent from the surface of the coating; the size of the bioactive agent; the hydrophilicty of the bioactive agent; and the strength of physical and physical-chemical interaction between the bioactive agent, the bioactive coating and/or the mesh material. By properly controlling some of these factors, a controlled release of a bioactive agent from the mesh of the present disclosure can be achieved.

In embodiments, filaments utilized to produce the strands of the mesh implant of the present disclosure may be made of bicomponent microfibers. Bicomponent microfibers typically include a core material and a surface material. In embodiments, the bicomponent microfibers may include a nonabsorbable or long lasting absorbable core and a shorter lasting absorbable surface material. The surface material of the bicomponent microfiber may be absorbed by the body within a number of hours, such that only the core portion is left in the body for an extended period of time, typically for a long enough period of time to enable tissue ingrowth. Although a variety of materials may be used in forming these bicomponent microfibers, suitable materials include polypropylene for the core and polylactic acid or polyglycolic acid for the surface material. In another embodiment, the bicomponent microfibers may be made of a core material which may be rapidly absorbed by the body and a surface material which is not rapidly absorbed, but instead is absorbed for a longer period of time than the core.

In embodiments, the surface material of the bicomponent microfibers may provide the mesh implant with enhanced characteristics required for surgical handling. After insertion in the body, the surface material of the bicomponent microfiber may be absorbed by the body leaving behind the reduced mass of the core material as the strands of the mesh. For example, suitable bicomponent microfibers include a polypropylene non-absorbable portion as the core and a polylactic acid absorbable portion as the surface. The surface material is present during the surgical procedure when the mesh is being inserted and located in the patient, and provides the mesh with characteristics desirable for surgical handling. Following a period of insertion in the body, typically a few hours, the surface material is absorbed into the body leaving only the core material of the filaments in the body.

It may be desirable to provide a variety of implants having different sizes and dimensions so that a surgeon can select an implant of suitable size to treat a particular patient. This allows implants to be completely formed before delivery, ensuring that the smooth edge of the implant is properly formed under the control of the manufacturer. The surgeon would thus have a variety of differently sized and/or shaped implants to select the appropriate implant to use after assessment of the patient.

In another embodiment the mesh can be cut to any desired size. The cutting may be carried out by a surgeon or nurse under sterile conditions such that the surgeon need not have many differently sized implants on hand, but can simply cut a mesh to the desired size of the implant after assessment of the patient. In other words, the implant may be supplied in a large size and be capable of being cut to a smaller size, as desired.

Even where the cutting of the mesh causes an unfinished edge of the mesh to be produced, this unfinished mesh is not likely to cause the same problems as the rough and jagged edges of the implants of the prior art, due to the smaller diameter filaments and, in some embodiments, treatment of the mesh with a coating, which protects the tissue from the mesh during the surgical procedure when damage to the tissue is most likely to occur.

The minimal elasticity and shrinkage of the mesh implant of the present disclosure renders the implant capable of stretching to a limited extent in response to forces applied to it while in the body of a patient, and then returning to its original configuration upon removal of the stress to which it was exposed. This minimal elasticity means that a mesh implant of the present disclosure may, in embodiments, stretch no more than about 5% of its length in the direction to which a force of 5 Newtons is applied, in some embodiments it will stretch no more than about 2.5% of its length in the direction to which a force of 5 Newtons is applied.

Thus, for example, a mesh implant of the present disclosure that is 200 mm long, when subjected to a load of 5 Newtons in the longitudinal plane and held at this load for 60 seconds, should typically only stretch by about 4 mm to about 10 mm. One hour after removal of the load, the mesh implant should recover to a length ranging from about 200 mm to about 205 mm.

The minimal elasticity and shrinkage of the mesh implant of the present disclosure, in combination with its ability to return to its original configuration, means a patient with such an implant will suffer less tissue distortion following implantation of such an implant in comparison to conventional implants. Moreover, the minimal elasticity and shrinkage means the mesh implant of the present disclosure is capable of remaining flat, even under tension.

Medical implants of the disclosure may include, but are not limited to, incontinence tapes and slings, and meshes, patches and/or implants for use in fascial repair, hernia repair or prolapse repair. Different shapes are suitable for repairing different defects. Thus, by providing a mesh implant which can be cut to a range of shapes, a wide range of defects, including those found in fascial tissue, can be treated.

Where utilized to treat urinary incontinence, the mesh implant of the present disclosure is capable of being fixed such that, in use, the mesh implant passes under the urethra and, during periods of increased abdominal pressure, the mesh implant supports the bladder neck or the urethra. In some embodiments, the mesh implant may be located around the mid point of the urethra such that a small space exists between the portion of the mesh implant which passes under the urethra when the urethra is in a rest position, i.e., during periods of non-increased abdominal pressure. The mesh implant is smooth enough to allow for placement under the middle portion of the urethra, but possesses sufficient texture to grip the tissue adjacent to its placement, which helps keep the mesh in position.

In some embodiments, it may be desirable to secure the mesh in place once it has been suitably located in the patient. The mesh implant can be secured in any manner within the purview of those skilled in the art. Some examples include suturing the mesh to strong lateral tissue, gluing the mesh in place using a biocompatible glue, or using a surgical fastener, e.g., a tack, staple, tissue anchor, bone anchor, etc. to hold the mesh securely in place.

In embodiments it may be advantageous to use a biocompatible glue since it is fairly quick to apply glue to the area around the surgical implant. Additionally, the mesh may include at least one capsule containing a biocompatible glue for securing the implant in place. In certain situations the mesh may include up to about four capsules containing a biocompatible glue which may be provided around the perimeter of the surgical implant. The capsules may be hollow thin-walled spheres from about 3 mm to about 5 mm in diameter and may be made of gelatin.

Any biocompatible glue within the purview of one skilled in the art may be used. In embodiments useful glues include fibrin glues and cyanoacrylate glues.

In another embodiment, the mesh implant of the present disclosure may be secured to tissue using a surgical fastener such as a surgical tack. Other surgical fasteners which may be used are within the purview of one skilled in the art, including staples, clips, helical fasteners, suture anchors, bone anchors, hooks, and the like.

In embodiments, it may be advantageous to use surgical tacks as a surgical fastener to secure the mesh implant. Tacks are known to resist larger removal forces compared with other fasteners. In addition, tacks only create one puncture as compared to the multiple punctures created by staples. Tacks can also be used from only one side of the repair site, unlike staples, clips or other fasteners which require access to both sides of the repair site. Suitable tacks which may be utilized to secure the mesh implant of the present disclosure to tissue include, but are not limited to, the tacks described in U.S. Patent Application Publication No. 2004/0204723, the entire disclosure of which is incorporated by reference herein.

Suitable structures for other fasteners which may be utilized in conjunction with the mesh implant of the present disclosure to secure the implant to tissue are within the purview of those skilled in the art and can include, for example, the suture anchor disclosed in U.S. Pat. No. 5,964,783 to Grafton et al., the entire disclosure of which is incorporated by reference herein. Additional fasteners which may be utilized and tools for their insertion include the helical fasteners disclosed in U.S. Pat. No. 6,562,051 and the screw fasteners disclosed in International Patent Application No. PCT US04/18702, filed on Jun. 14, 2004, the entire disclosures of each of which are incorporated by reference herein.

The surgical fasteners useful with the mesh implant herein may be made from bioabsorbable materials, non-bioabsorbable materials, and combinations thereof. Suitable materials which may be utilized include those described in U.S. Patent Application Publication No. 2004/0204723 and International Patent Application No. PCT US04/18702, the entire disclosures of each of which are incorporated by reference herein. Examples of absorbable materials which may be utilized include trimethylene carbonate, caprolactone, dioxanone, glycolic acid, lactic acid, glycolide, lactide, homopolymers thereof, copolymers thereof, and combinations thereof. Examples of non-absorbable materials which may be utilized include stainless steel, titanium, nickel, chrome alloys, and other biocompatible implantable metals. In embodiments, a shape memory alloy may be utilized as a fastener. Suitable shape memory materials include nitinol.

Surgical fasteners utilized with the mesh implant of the present disclosure may be made into any size or shape to enhance their use depending on the size, shape and type of tissue located at the repair site for attachment of the mesh implant. The surgical fasteners, e.g., tacks, may be used alone or in combination with other fastening methods described herein to secure the mesh to the hernia, prolapse, or other repair site. For example, the mesh implant may be tacked and glued, or sutured and tacked, into place.

The surgical fasteners may be attached to the mesh implant in various ways. In embodiments, the ends of the mesh may be directly attached to the fastener(s). In other embodiments, the mesh may be curled around the fastener(s) prior to implantation. In yet another embodiment, the fastener may be placed inside the outer edge of the mesh and implanted in a manner which pinches the mesh up against the fastener and into the site of the injury.

In some embodiments, curved, needle-like elements may be connected to each end of the mesh implant, similar to those found on the TVT Tension-free Vaginal Tape. Unlike the TVT Tension-free Vaginal Tape, the mesh implant of the present disclosure does not require a removable plastic sheath, due to the minimal elasticity of the mesh implant of the present disclosure. As described above, other fixation devices may be attached to the mesh implant at its ends to facilitate insertion and attachment of the mesh implant of the present disclosure.

By suitable location of a mesh implant to support the urethra at times of increased abdominal pressure, the voiding of urine during moments of physical stress including coughing or sneezing can be minimized. The mesh implant supports the urethra by strengthening weakened or damaged muscles, which control urination. The mesh implant may additionally facilitate the repair of damaged tissues.

A variety of different surgical approaches are contemplated herein for introducing the mesh implant of the present disclosure into a patient, including supra-pubic (i.e., the distal end of a needle initially being inserted through an abdominal incision and then emerging from a vaginal incision), trans-vaginal (the distal end of an insertion needle being initially inserted through a vaginal incision and then emerging from an abdominal incision), trans-obturator (e.g., the distal end of a needle initially being inserted through an incision in skin near the patient's obturator foramen and then emerging from a vaginal incision or vice versa), and posterior approaches. The implants according to the present disclosure may, in some embodiments, be inserted through a vaginal incision. Alternative insertion routes such as laparoscopic and through an open abdominal incision are also within the scope of the present disclosure.

Methods for treating urinary incontinence with a mesh implant of the present disclosure are within the purview of one skilled in the art. In one embodiment, there is provided a minimally invasive method of treating urinary incontinence, which may include the following steps: providing a mesh implant of the present disclosure; transvaginally introducing the mesh implant into the patient's body; advancing at least two fixation devices through the vaginal mucosa and into an internal support structure or tissue to attach said mesh implant to said tissue and elevate the bladder neck or urethra; advancing the at least two fixation devices through the vaginal mucosa and into an internal support structure or tissue; and attaching the fixation devices to the patient's internal support tissue, thereby retaining the mesh implant in a position capable of supporting the bladder neck or urethra. The internal support structure or tissue to which the fixation devices are attached so the mesh implant supports the bladder neck or urethra may include ligamentous tissue, a iliopectineal ligament, a bone, including the pubic bone, and any other internal structure which may be utilized to suspend the mesh implant of the present disclosure so that it may support the bladder neck or urethra.

According to another aspect of the present disclosure, there is provided a minimally invasive method of treating uterovaginal prolapse which includes the following steps: making an incision in the vaginal wall close to the opening of the vaginal cavity; making a subcutaneous cut, through the incision, over and surrounding the area of the prolapse, which cut is substantially parallel to the vaginal wall; and inserting a mesh implant according to the present disclosure through the incision, into the space defined by the cut.

Thus, a mesh according to the present disclosure can be inserted through a small incision (e.g., about 1 cm to about 2 cm in length) in the region of the periphery or opening of the vaginal cavity. An incision in this position is easier for a surgeon to access than an incision deeper in the vaginal cavity. It is also more convenient to treat a vaginal prolapse by implanting a mesh of the present disclosure through such an incision.

In one embodiment, the incision may be at the anterior or posterior extremity of the prolapse sac of the vaginal cavity. This may be desirable, as prolapse most often occurs in the anterior or posterior vaginal wall, so positioning the incision in such a location allows the most convenient access to these parts of the vaginal wall.

The mesh implant of the present disclosure may also be introduced into a patient utilizing a surgical device, sometimes referred to as a tunneller instrument. Suitable tunneller devices include the OBTURATOR IVS TUNNELLER™ IVS04 from Tyco Healthcare UK, Ltd. In one embodiment, the tunneller instrument may be used to insert a mesh implant of the present disclosure configured as a tape into the body to support the upper level of the vagina. The tunneller has an outer tubular member including a longitudinal proximal end and a curved distal end and a stylet movable within the tubular member and configured to hold an end of the length of material, that is, the tape. In a procedure to correct urinary incontinence or vaginal prolapse, the stylet is positioned within the tubular member.

The use of the tunneller to transvaginally insert a length of tape beneath the mid line of the urethra to support the urethra includes inserting the instrument through the obturator foramen, passing around the internal rim of the ischiopubic ramus, and exiting via an incision in the vaginal wall. Initially, the vagina is grasped and an incision is made in the anterior vaginal wall. Dissection is made laterally from the vaginal incision in order for the ischiopubic ramus to be palpated. A skin incision-located-lateral to the vulva, in line with the clitoris, and above the obturator foramen is made on both sides. The device, in embodiments a tunneller device such as an OBTURATOR IVS TUNNELLER™, is inserted through this incision in a vertical orientation. With a finger inserted in the dissection plane, the device is oriented towards this finger, thus penetrating the obturator membrane and fascia. The handle of the device is inverted in a 3 dimensional orientation in order for the tip of the device to exit via the vaginal incision. The stylet is inverted. The mesh sling is threaded through the stylet, and pulled through the tunneller device. The same procedure is then performed on the opposite side. The vaginal incision is closed with suture. The tension of the mesh sling is adjusted, the ends of the tape are sectioned subcutaneously, and the two skin incisions are closed.

Embodiments of the present disclosure will now be described, by way of example only, with reference to the accompanying drawings.

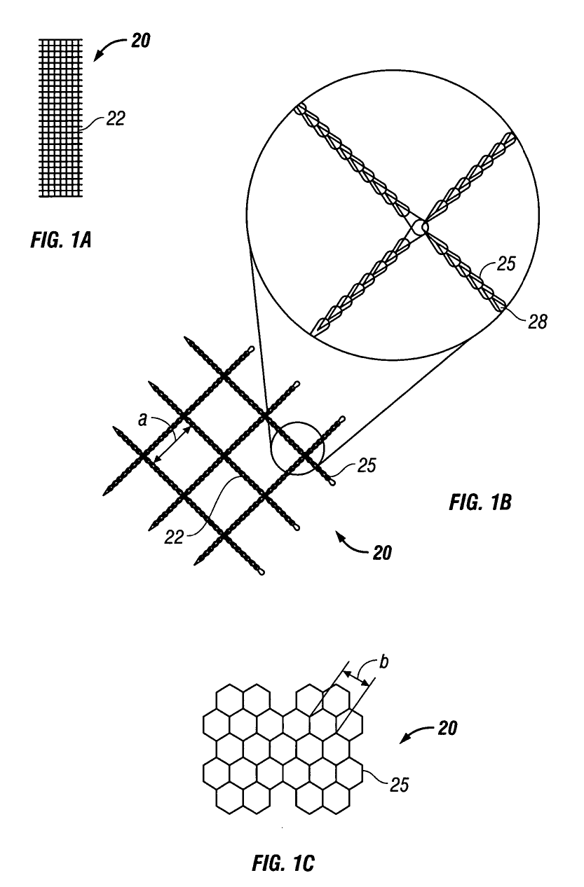

As shown in FIG. 1A, the mesh implant of the present disclosure may be a flat tape or sling 20 comprised of strands 22. As depicted in FIG. 1A, the pores of the mesh implant may be in a square or box-like configuration. In other embodiments, the pore of the mesh implant may be in a triangular configuration depending on the weave design. The strands are arranged such that they form a regular network and are spaced apart from each other such that the pores formed between the strands may be from about 200 microns to about 2000 microns in the mesh. In one embodiment the mesh portion of the sling is about 500 mm in length, about 8 mm in width and a thickness of less than about 0.3 mm. This mesh has minimal elasticity and shrinkage, and is capable of returning to close to its original configuration upon removal of any stress.

Turning to FIGS. 1B and 1C, a mesh implant for use in treating urinary incontinence may have a diamond shape weave or a hexagonal shape weave, respectively. As depicted in FIG. 1B, the mesh 20 is comprised of strands 22. The strands are arranged such that they form a regular network and are spaced apart from each other such that, for a diamond shaped mesh, the strands form pores of from about 200 microns to about 2000 microns in diameter in the mesh. In a hexagonal net arrangement, the space is similarly from about 200 microns to about 2000 microns between opposite diagonal points where the strands of the mesh interact as depicted in FIG. 1C.

As depicted in FIG. 1B, the strands 22 may be produced by weaving a monofilament 25 to produce minute openings 28 in the strand 22. In the embodiment shown in FIG. 1B, the filament 25 of the strands 22 is knitted using a warp knit to reduce the possibility of fraying of the filaments 25 and strands 22.

Other methods of reducing fraying of the filaments are heat treatment, laser treatment or the like, to seal the edges of the surgical implant. In some embodiments a heat treatment may be desirable, as such a treatment promotes adhesion of the strands forming the mesh to each other, thereby facilitating removal of the mesh implant if required for any reason.

The mesh 20 may be supplied in any shape or size and cut to the appropriate dimensions as required by the surgeon.

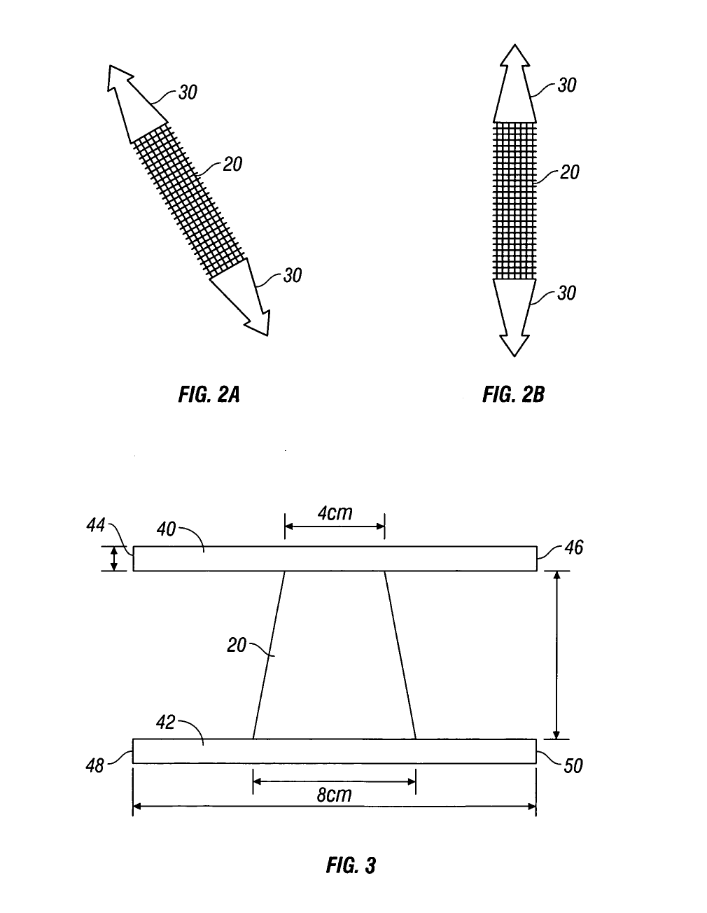

As shown in FIGS. 2A and 2B, the mesh implant of the present disclosure may be a flat tape or sling 20 affixed to two fixation devices 30, the fixation devices capable of achieving multilayer fixation in the paraurethral space such that in use the sling 20 is positioned loosely under the urethra.

As shown in FIG. 3, in another embodiment of the present disclosure, the mesh implant may have a trapezoidal shape suitable for treating conditions such as anterior vaginal wall prolapse (cystocele). As depicted in FIG. 3, a trapezoidal mesh implant may, in one embodiment, include a tape portion 20 affixed to two anterior and posterior support extensions 40, and 42, extending to about 20 cm by about 8 cm. Anterior and posterior support extensions 40 and 42 may be made of the same material as tape portion 20 or a different biocompatible material. Each end of the anterior and posterior support extensions, that is 44, 46, 48 and 50, may be utilized to affix the mesh implant in the body.

In embodiments, a tunneller device described above may be utilized to introduce a mesh implant having a configuration depicted in FIG. 3 to treat cystocele. The method for introducing such an implant is similar to the description of use of the tunneller device above, however four insertion points may be prepared as follows (skin incisions and entry points of the tunneller device) for each end of the attachment arms 44, 46, 48 and 50 above the obturator foramen, and then passing the arms 44, 46, 48 and 50 through the arcus tendineous as anchor points and out through the vaginal incision.

In one embodiment, an implant having a configuration depicted in FIG. 3 may be utilized as follows to treat cystocele. A sagittal incision is made in the anterior vagina. Two skin incisions are made on each side of the vulva: one at the crossline of the external margin of the ischiopubic ramus and the horizontal line at the level of the clitoris; the other at the external margin of the ischiopubic ramus as close as possible to the ischion. The distance between these two incisions is usually about 6 cm.

A tunneller device, such as an OBTURATOR IVS TUNNELLER™ device, is positioned at the anterior incision, with one finger palpating the ischiopubic ramus via the vaginal dissection, at the level of the bladder neck. The handle of the device is rotated medially in order to bring the blunt tip towards the finger positioned in the dissection. When the blunt tip is correctly positioned, the obturator muscle is perforated, direct contact with the finger protecting the bladder neck is obtained, and the tip exteriorized. The plastic stylet is then reversed. One anterior arm 44 of the trapezoidal cystocele repair mesh is inserted into the stylet and drawn through the tissues. The same procedure is performed contralaterally for arm 46. The tunneller device is positioned at the posterior incision vertically, the tip is inserted through the obturator membrane but not through the ilio coccygeous and the obturator muscles. The tip is positioned medial to the ischial spine, above the arcus tendineus, the muscle is perforated, and the tip exteriorized through the vaginal incision. The stylet is reversed, and one posterior extension arm of the trapezoidal mesh 48 is inserted into the stylet and drawn through the tissue. This is repeated contralaterally for arm 50. The anterior and posterior supporting extension arms are adjusted appropriately, applying the correct amount of tension, and the vaginal incision is closed. The four ends 44, 46, 48 and 50 of the extension arms of the trapezoidal mesh are sectioned subcutaneously, and the skin incisions are closed.

While the above description contains many specifics, these specifics should not be construed as limitations on the scope of the disclosure herein but merely as exemplifications of particularly useful embodiments thereof. Those skilled in the art will envision many other possibilities within the scope and spirit of the disclosure as defined by the claims appended hereto.

What is claimed is:

1. A medical implant comprising:
strands having a maximum residual mass density of from about 30 g/m² to about 60 g/m² in a mesh configuration; and
pores of from about 200 microns to about 2000 microns in diameter in the mesh.

2. The medical implant of claim 1, wherein the mesh comprises a multifilament mesh.

3. The medical implant of claim 1, wherein the mesh comprises a monofilament mesh and the pores are from about 500 microns to about 1500 microns in diameter.

4. The medical implant of claim 1, wherein the strands comprise a synthetic non-absorbable material selected from the group consisting of polyethylene, polypropylene, copolymers of polyethylene and polypropylene, and blends of polyethylene and polypropylene.

5. The medical implant of claim 1, wherein the strands comprise an absorbable material selected from the group consisting of trimethylene carbonate, caprolactone, dioxanone, glycolic acid, lactic acid, glycolide, lactide, homopolymers thereof, copolymers thereof, and combinations thereof.

6. A medical implant comprising:
strands having a maximum residual mass density of from about 30 g/m² to about 60 g/m² in a mesh configuration;
pores of from about 200 microns to about 2000 microns in diameter in the mesh;
the mesh having a thickness of from about 0.2 mm to about 0.4 mm; and
a bioactive coating.

7. The medical implant of claim 6, wherein the mesh comprises a multifilament mesh.

8. The medical implant of claim 6, wherein the mesh comprises a monofilament mesh and the pores are from about 500 microns to about 1500 microns in diameter.

9. The medical implant of claim 6, wherein the strands comprise a synthetic non-absorbable material selected from the group consisting of polyethylene, polypropylene, copolymers of polyethylene and polypropylene, and blends of polyethylene and polypropylene.

10. The medical implant of claim 6, wherein the strands comprise an absorbable material selected from the group consisting of trimethylene carbonate, caprolactone, dioxanone, glycolic acid, lactic acid, glycolide, lactide, homopolymers thereof, copolymers thereof, and combinations thereof.

11. The medical implant of claim 6, wherein the bioactive coating comprises a bioactive agent selected from the group consisting of antimicrobials, analgesics, antiadhesive agents, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, narcotics, lipopolysaccharides, polysaccharides, polypeptides, proteins, hormones, enzymes, and combinations thereof.

12. The medical implant of claim 6, wherein the bioactive coating comprises an absorbable material selected from the group consisting of gelatin, starch, cellulose, alginate, hyaluronic acid, trimethylene carbonate, caprolactone, dioxanone, glycolic acid, lactic acid, glycolide, lactide, homopolymers thereof, copolymers thereof, and combinations thereof.

13. The medical implant of claim 6, wherein the bioactive coating further comprises a fatty acid component selected from the group consisting of fatty acids, fatty acid salts, and salts of fatty acid esters.

14. A method of treating urinary incontinence comprising the steps of:
providing a mesh implant comprising strands having a maximum residual mass density of from about 30 g/m² to about 60 g/m², pores of from about 200 microns to about 2000 microns in diameter in the mesh, and a thickness of from about 0.2 mm to about 0.4 mm;
transvaginally introducing the mesh implant into a patient's body;
advancing at least two fixation devices through the vaginal mucosa and into an internal support structure or tissue; and
attaching the fixation devices to the internal support tissue, thereby retaining the mesh implant in a position capable of supporting the bladder neck or urethra.

15. A method of treating vaginal prolapse comprising the steps of:
providing a mesh implant comprising strands having a maximum residual mass density of from about 30 g/m² to about 60 g/m², pores of from about 200 microns to about 2000 microns in diameter in the mesh, and a thickness of from about 0.2 mm to about 0.4 mm;
transvaginally introducing the mesh implant into a patient's body;
advancing at least two fixation devices through the vaginal mucosa and into an internal support structure or tissue; and
attaching the fixation devices to the internal support tissue, thereby retaining the mesh implant in a position capable of supporting the bladder neck or urethra.

16. A method of urinary incontinence comprising the steps of:
providing a mesh implant comprising strands having a maximum residual mass density of from about 30 g/m² to about 60 g/m², pores of from about 200 microns to about 2000 microns in diameter in the mesh, and a thickness of from about 0.2 mm to about 0.4 mm;
providing a surgical device having an outer tubular member including a longitudinal proximal end and a curved distal end and a stylet movable within the tubular member and configured to hold an end of the length of material;
positioning the stylet within the tubular member;
making a transvaginal incision and a lateral skin incision above a patient's obturator foramen, lateral to the patient's vulva, and located over the obturator foramen;
passing the curved distal end of the surgical device through the incision over the obturator foramen;
manipulating the surgical device such that the curved distal end passes through the obturator foramen and out the transvaginal incision;
engaging a proximal end of the stylet with a first end of the mesh implant;
reversing the stylet; and
drawing the stylet through the tubular member to draw a portion of the mesh implant from the transvaginal incision to an exit point at the incision above the obturator foramen following reversal of the stylet.

17. The method of claim 16, wherein the outer tubular member is withdrawn through the incision over the obturator foramen leaving the mesh implant extending through the obturator foramen and out the vaginal incision.

18. A method of treating vaginal prolapse comprising the steps of:
providing a mesh implant comprising strands having a maximum residual mass density of from about 30 g/m² to about 60 g/m², pores of from about 200 microns to about 2000 microns in diameter in the mesh, and a thickness of from about 0.2 mm to about 0.4 mm;
providing a surgical device having an outer tubular member including a longitudinal proximal end and a curved distal end and a stylet movable within the tubular member and configured to hold an end of the length of material;
positioning the stylet within the tubular member;
making a transvaginal incision and a lateral skin incision above a patient's obturator foramen, lateral to the patient's vulva, and located over the obturator foramen;
passing the curved distal end of the surgical device through the incision over the obturator foramen;
manipulating the surgical device such that the curved distal end passes through the obturator foramen and out the transvaginal incision;
engaging a proximal end of the stylet with a first end of the mesh implant; and
drawing the stylet through the tubular member to draw a portion of the mesh implant from the incision over the obturator foramen and through the transvaginal incision.

19. The method of claim 18, wherein the outer tubular member is withdrawn through the incision over the obturator foramen leaving the mesh implant extending through the obturator foramen and out the transvaginal incision.

20. The method of claim 18, wherein the mesh implant further comprises two attachment arms having four ends, the four ends each being attached to the stylet and drawn from the incision over the obturator foramen and through the transvaginal incision.
